SEQUENCE LISTING (3 sequences)

SEQ ID NO: 1 - DNA, 12300 bp, Bos taurus
tcttagagaatgaaaaagaaggctggctcttcaaagtctgcaatagcatttttaaatgtatatataagctgttctgaggacccagcagttgtgcagctctctggagctaatagtaaaattaagtgttagttgcagccgccacaggtgtaagcactattaaagtagtgttccttcattcactgttgcccatttactgagtactgagagacttcactttcacttttctctttcacgcattggagaaggaaatggcaacccactccagtgttcttgcctggagaatcccagggacgggggagcctagtgggctgccatctctggggtcgcacagagttggacacgactgaagcgacgcagcagcagcagcagcagcagcagcaacagcagcaggaaaatacagatggaaacatgaataagacccatatggactcaagcatagcacccacgcattgtattgacaaacaagaactatatgctcagaattagttgctgtggatggacagttgagtgcttactcattaacagtttattgaatatttatttactctataacaactctatgggtctaaaatgaaacttttttaagtgacattaatgtttaccagtagactattttttaaggagctgaattttgttgcctaatttattgagcaataggatacaaagtttgggcgattttttttttgtatttctgaaatattctaatttcatgtcttaacttttaagaaaacttcaaacgagttcctttttatggctgagtaatattccattgtatgtatgtaccacaacttctttatccattcatctgttgatggacatctaggttgcttccatgtcttggctgttgtatacagtgctgcaatgaacattggaggacatgtgtctttttcaattatagttttctcagggtatatgtccagtaatgggattgctgggtcataaggtagctttattcctagtttttaaagtattatccatactgctccccacagtggctgtatcaatttacattcccaccaacaatgccagagagttcccttttgagtcaggtatagtgaggtggatgaacctattgcctgttatacagagcaaagtaaatcagaaagagaaaaacaaatatcatacattaacgtatacatatggaatctagaaaaatagtactgatgaacctatttgcaggagaggaaagatccagacatagataatagacttgtggacacagtggtggaagaagagagtgggacaaattgagagggcagcattgaaacacacacatggccatatgtaaagcaggtagttaatgggaagtcactgtataacacagggagttctactcggtgctctgtgacaacctggaggggtaggatgggggaggggggaaggctcaagagggaggggatatatgtatacttatggctcattcatgttgtatggcagaagccaacaaaatatcgtaaagcaattatccttcaattaaaaataaattgaaaaaaaacccttatcatcaacccttgcttccctccaataaataattcccttcttccctagactacatatgaataaataattgaatagtaggcttccaatgaaatataattcccaacttctcaatgacagatagcaatggcacaagatcagctttttatctgaacctttactcaagggaaaaaatatttacagagctgaaaaagtcaaaattgttgattatacttagtaaaatataattacaaatatccaaataaactagaagtcaatataataaagtccaccaaaactggatacaactggacacaaattttcttattatatgaaaattttcatgtaatttgtgggaagaaaacataatgttctttatgcctccatacatcctcttagagtgtttatattttatgatcacaactcattctccatttcagtttaacactaaatttattccctttctggatacaaattttacaaggaatttgatcatgttgaccttttggttatatcaaccaaggattaaactaattcaagaatggaaatagtgtggaaatgctacagatcaagcctctctgattcaagtcattcaacatgctaattctgacagctggaaaaccttactcagaaaaaaaataagtgtcagcagcatgtttgactatgaaaagctggaagcaggagtctcttcaaaaatcagaatggggatttctcagtgtcttagcgtcaagaacccgcctgccaatctaagagacccgcaggtgcaactaatcctgtgagccacaactactgaacctgtgctctagagcggggggaactgcaaataccgaagcctgagaactctagagcccgagctccgcaacaagagacgccactgcaatctgaagcgcaggcattacaactagagaaaagcccagcagcaacaaagacccacctcggccaaaaataagtaaaattattcaaaataattagaatgacgcactatacatctcatactttacagaaccgtctctaccacaaaccattttatcctctcatctctgaacatgccagacgtgttctttaaccatgaattcacaagcctttactctgagtaagccttgcgatcagtaacctcttgagcgagtgtgttaaacagggaaggagtccatagcttacacctggggtcgccgaagacaacacagcagaaaagaatgtttttcttctcgtggcctctgtgtcccctgttaatgtgtagcgggtcattgttgggaaatacatcctcacctgacattccaagaaatggagggcctcacggttgttttatggctcagtcactaaatgcatgaacgttccatcctgtcggaaatcactgacgcagccaggctgtgaagtccttctcagaaacgctgccaccgtgtggtaatttggaggtatgaccagaagagccgtgtgggtcatatggttcatctcaagatggctgggctccagcagaatctattcctataactttctaacttcctattaaatcattcccgtgggcacagagtaaaccatagtgaatcctgtggaaatcttggggtttttttttaggatgtgtgcgcttccctccactacattgacaatgtgacagttatgcgggtaatgtgtttaaagggaaacaacagtttttaagagcagaaataagaaatctgtttttgcaactttataacctatgtgttttatattttatacagatagctgccaattgagttgttaaaatgtgtgtttagaacccacaaatcaaaaacatcaatttatttgaaatgcatccaatttaaaagagacatattccacctgctccccaccctcagcccaccaccaaatacctacaaacctatggatttaatcaattaaattttgattttttaaaaaattattttttattaaataatcaaaacattatttaattaatatgtatattaaaagcaaaattgggggaacttctctggtgccccggggtctaaggctccacactcccaatgcagggggcccggatttgatccctgatcagagaactagatcccacatgccacaactaagagatcccacatgcacctagcacctggtgctgccaaagaaaagttttctaaaaagcaattttggtaaataggagctgacatcctacatagggccatttataataaataggctattataataaatagggccatttatctttactctcaccttttgcatgattcttacaatggaagcgtgagataaatgaatagtgcaatctccatttcacaactgagaaaggtagatgaagaggttaagtaatcttgaaacaatattaaatgtttaaaatgaactcagagctctgctacccctaacttctgttccaatattcaaccttcatccataattttctttcaaacaccttttaaatgcccattaaagttttttttttaatatagaatttttattttcttattcagtaaccaattttatatatcctgagagaaaaattagaaaatgacaattaagaaatctaagccagtccttcagagacatgcaaattatcctgttgacatacagtataaaaatcttatatccgatctcattacaataaaccattccatttagagttaatacaaatcatgactacctttttctcctaaaaatcttaataattgttaacatacaattaaatatggttaaaatatgcagggtatttgcaaatatgtgggaggtatttttagttttacacattctaattcacttaaatctctcaaaaaccccacgaactctgcatttgacagatgaagaaacaagtatagataggctaaatgatttgcccaaggtcacacacctaatttgtgcagagtagaattcaaagccacgtgctggctcctgaaacagcactttcaagcagtaaccctgctcggtcataaactgctttacttctgccacactgagaataaattaccattcgtttattttatatagcagttatcagatccatcagctccaaataattcaaaccatgcaacaaaaatccatgtccttgaaagtcattgtgaatccaatttaatgaacgtgactggggattttaatgtttctaataccatttgtctacatgacaagctgctgcacacttgaaactcaaaatgaagcttaagtaaaaatggaggaagggtatctgaactctttgtatgtatctgaacaatagcctggtgcctcttaaataagtataattccgttgcatatatcacattaatctgttatttaaaccaaaagaaacaagactattctatttaattttatctaggactaatttagataatattttcttctaactcagaaataaacccttttccctccctctacgttttcatgggccctctagatgcccttccaggatgctggaagtcagtgctatgaacaaaaaaagatagttagtgatattgtacataagtaatgttttaactttaactagcagggtagtgggtgtttgtgtgcgtgtgcgcgtgtgtgtgtgcctgtgtttgttctgtgaccacaaaaccagagggggaagtgtgggagcaagtgggctgggtagtggcaaaatgccccatgacacatctctccgccccctgtgtaggtggagagcatctggagcagcctttaaattctgggagatcctggttgtcagcagcagggagagcaggccaggagggcagcactgagcactgcatcagcatcacaggggactggactcttctcgccgccgcagaccaaggtaagcctgcagtttgctacagactcctgtcctctctgtgcgctgcctcatttcattgggaaggtcaatttgtaaggaaaagagtataatggtaaacactgttaatcagacttgagttgttcttgtcttttgaatatgcatgccagatccagggctgatgtcctgcaggaaaggtggccggttattttgaaagacagtcgaatataaaacttgaaaatatttccatggagtcctcaaaagaattgagactacttttttcagtcaggaaataaaagaaaaattctatgcccttttgggatgattgtatatacatcattttaatgaatagatgaccatgggatatttaaaaggaaaatgctttttagtattcatataacctgacgttaatgctaatttttagtgatttgatttatccctttttcaaggaaaaaaaaaaaaaccctttctgaatattttcacctctgtatttagctattaaaatttcacccaaatatctatatgatactgtttagacttacaaatagaaaagctgttgacttcagtgttttctttttcatttcaaacttttagaataccttgacttactaaccttagagacagctacattacacctaactaataccttttaaataatttaaaatcacatttgaaatgcatgttggaaaatggagacagcaagtttctctttcttatttttatcttctctcttcatgtttttcttctctgaaaagtaaatattctcattcttgctttattattttaattcaattactgctgatctgtttttaggtttagatagctggagatatcaggtagtaatggtgtaatctctgaaactctaaatgttaaagtcgaataaatatagatttgtaaaattcctctctcccttgcctaatagtgagagatggaaaatagaggtggcagtacaaatattaactcaaaagatcataatattaaaaagaaattagtggagtgtttccacacaaaatacatatttatttgtgatgattttgtaatgtggtagcctaaaaaaagtatcactgttttgaccttagaaaagataaaatatttcttacaaaatattttgcaggaaaaatcattaccatgagaattgcagtgatttgcttctgcctcttgggcattgcctccgcccttccagtgagtacaactgaatcttttttttttttaaatgtccttaaaataaatgaattgtgtgtttcaatgttgttaggaacacattcacactgtaatctttcttcacctttctatttcaaaggttaaaccgaccagttctggcagctctgaggaaaagcaggtaagcatctttttggtttattatatagttatttatgcaacttctgcaagatgtactacaaacatatgacaattttacttatttgcacaaataaataactgataagtatttattgtgtgcctgctatgcttctagtgcagtaccaagcatagaattgcttttattatacagtctcttaaactaaactagatttcttaatacaataaaatgatgctttaggtcaatcatttctataaaatgagttctgtgaagttgtgtgacttttatttccatgagtcaacttagttctagaagtaaaaaaatccatctttcactttacttaatggcaagctaagtttaaaattcacttcacgtattggagagttggcataaccataatattcacagttatctctgaagatatttgctaagaaacccattatctttaaaaaaaaaaacacagcatatttatgctgtttgtatctattgggcttatcagatgactttagatttttctaaccaaacataagaccaaatcaaactcttcataattattccacccagcaccatagatgtacctgtccctccacaataggtgaaaccatgccagccaaacaacaaacgggtattgtcccaaggagcttgaacaaaaaggcacacagggttcaattccagataaacagaataaaggccaatatagagctgtctgaggtcactgcagttagattgctcaatgaagatgttaggagaagtactgctgtgggtgtttggatatgcaaataggcatgtgtaagtgtgtctgttttccaactgatctaagaacacaagtattgaatcacaataataagccagaaaattaacatttccagagaaaatagaggtcacactctttaaggcaagaattagtgcctttttaatagaataaaatatggcacatgaagctatggcagtgtaagtggttaaatgttaattaagagataagtggagagcagaaccttctagaatcctagaaatgtagagctagtaggaagtatggaaagtctataaagagacacccctgaagatgaaaacactggttatctatgaatgatatgattactgagcaattttagtttgtttttcttatctgtatgttcttcatattactttttaaaaattataaatttatcttataaatacagataaagaatgattacatctatcagaaaatatattaattatatatcaattataacatttctgtaaatattatatatatatttatataaatatatattttaaagaaaaaaataggaacctttgaactcatctacagcaatagttttcacattttttagttgaagaaaacccttctttcagcaaaaaccttcatagaagtccaatatgaaactgctctggttagagccagacatgggaaacccaacatgctactaactagtttccttcctttcttcccctccttgcctgggaaagaattcttggaacccctagggtaactcttctcagtgacagagatgtgaagtgattgccttgggccatgcagcccaccagatgacacttattcagagcaatttccactcctctgcactagcatcttattgccatttcccagataaataatttggctgagtgaaagaccattccattcatgtctttggcagcttacagcaagagaacttttatgcaatggtgttgatactatatttctgcactgtccacaggtagccataaacacatgtgactgttggacactcacaatgcagctagtgccagagaagatagttttctttattaacttaaatagccatgtgtttcatggctagaggataccattcacgacagcacagcctgagagtttcttcttccatagaggaaggaaaaaaggaagaccagtgctaattaagggcacctctgctttagttgtgtttcatctgaggaatttcagctatcagacatagaacaggaaggcagtgtatcatcagtgtgaggagccattggatttaaaccctggctctgctacttactatcacttaactttattgtgggcttcccatctggtggctcagatgctaaagaaatctgcctgcagtgcaggagacccgggcttgatccctggatcagtcaggaagatcccgtggagaaaggaatggctatctactccagtattcttacctggagaattctgtggacagaggctgaaggaataataatgatgatgatgatactcatctcaggattaaatgaattagtatttataagtcactttgaaagtgtgcctgacttacagtaagtgctgttaaatgaatctatcagaaagaataaattatgaaaggtatcactaaggttctaattgactaaataatgcatattatgcttatttgtcaagattggagaagaacagttgttatttcagcttatcacttagagacccctgtttctttaaaaaataagaatgacaaatatgcaactctcttttgtagcttaacaacaaatacccagatgctgtagccacatggctaaagcctgacccatctcagaagcagactttcctagcaccacaggtatttttcattttaattaactctaaatattaaaattctcacaattaaagaacaaccactccaaaaaatagccaccaagcaggccatttgggctggttaaatggatcttccctgcctgttgggcttccctgatagctcagttggtaaagcatctgcctgcaacttggaagacccgggttcagtccctgggtcgggaagactccctggagaaggaaatggcaaccccctctagtactcttgcctggaaaatttccatggactgaggaccctggtaggctaagagtcagacagaactgagcaacttcacttcactttcctgcctgtttgtaaaagtgagcttaggacaccaattgatctgtcaggttgtcttccggcttaatccttccacaatgaggctagaaaaataagacctgctttggatggaaacagctaacttttgaataaaaaagttacgttgtatgatgtgcactgatttgtgtcttttcttcttcagaattctgtgtcctctgaggaaactgatgacaacaaacaaaatgtgagtctttgctttgattctgatgtctgttgtgccttagactcaggaaggcactctttctcctaatgacattgcccaggttcaaattccggcaaaattccactagcaaacccttcaggaactactttttattgggactattaatagggataagttaaatttgctttccttaagattctatttgaagatgctgagaatctataagagaagttagataaatgacccaggatatttgcaaatcagaagtgtgatagacattaactgagctatagtttctacacatggataagagagtcaccttttgattatccaggctaatagggaggtgattttagttttgggggtgtgcattaatacatggattctctgatcccctgagaattttcatttcaaatagaaaaggtagtctcacaattatgtatctgtatttattggatcattgaaatttggtaaattagtgtttattatgaacaaggaaaaacagtgtcattgatacaaatattataactcatacgtttggcttgaaaatatctgtgaaaatcgtttttatgagaaaccaagaaaaatgccttagaataggattccatttacccttgtgttaaaggggaaattggaataagctcattttagcatttaaaagccattaagtgctttgttgtgaatacaaagattctaaaactaaataaagatagtaaaatactaatgcactgtaaagcctaagggacagtaaaaaccctgacacccatttttctggccatcttgatttctagaccctcccaagtaagtccaatgaaagccctgagcaaacagacgatctagatgacgatgatgataacagccaggacgtcaactctaatgactccgacgacgctgaaaccactgatgaccctgaccattccgacgagtctcaccattctgatgaatctgatgaagttgattttcccactgatattccaacaatcgcagttttcactccgtttatccctacggaaagcgcaaatgatggccgaggtgatagtgtggcttacggactgaagtcaagatctaagaagttccgccgatctaacgttcaggtaaatcttgaatagacacatcagatggttatgattagagcccaattctaggaaactagagtctgcgtgctcacgtacgtgttcattcagcaactattattactttaagatcactgaatacaaaacctttcccacttggtttttgcgctcaagtccatataatttctccttaattttgtttctcaatcacaatgcaatttttccaaatcctgcttataaagcacttattgtattcactttttttaattaaaaaagactttggaaaagagtaattattttcacaatatagtggtcatatcttgaagttaaacacataagaactggagagttgcaaatcacactgactttatagtcaaaggatctggatatggcacatagccttgccatttactattgactttacttttaactttcgatccattttctctttatgtaaaacagataatgagacctacctcatagatttcctcatatacataattcatgtacttattaaactaaaggagatgacataatgaaagaacttggttttttaaaaggtagcatgctagtattttcttttctcttctatttttgcttcagccaaatttagacatttttatagactttggtgtggaagttagaaggcattagagaattacagtgcttcccttcctagctgttcgttgaattcttctcctgtttgtgctgtgattcagagtccagatgccacagaggaggacttcacatcacacatagagagtgaggagatgcatgacgcacctaagaagacgagtcagctgactgaccacagcaaggaaaccaacagtagcgagctttccaaagaactcacgccaaaggccaaggataagaacaagcattccaatctgattgagagtcaggaaaattccaaactcagccaagaattccatagccttgaagacaagctagacctagatcataagagtgaagaagacaaacacctgaaaattcgtatttctcatgaattagatagtgcctcttctgaggtcaattgaaaggagaaaatacaatttcttactttgcttttagtaaaaagaaaaggatacattaaagcagggtgggagacaatatgaaatgcatatttctcagcttagttggtgaatgtatatgtgtgtagatctggaaacagatcatgtttttgatcattagtttaatgtgtggcttcatggtaacacccttctaaactaaaagcttcagagtttagtctatgttctttccacatacaaaatgcaaaccatcacagcattttaatgtttgctacccttttaggaatagaaattcatgtagaagcaaacaaaatactgttacacacttttaagaaagaatataaaatttcatgtcactatgatcttttgttttttaaattagtatatattttgttgtgattattttttctgttgtgaataaatcttttatcttgaatgtaatgagtctggtgctatcagttgtgtttcttatttggtttcctacagttgcctagcaattataaatgtaatcattttaattacattaatatgctggacataatagagagcaatctaagaccttctgggtcagttatacagactctgactgagctaaagttcccgtcgagg

SEQ ID NO: 2 - DNA, 25 bp, Artificial Sequence
  Description of Artificial Sequence  Synthetic Primer
tccataattttctttcaaacacctt

SEQ ID NO: 3 - DNA, 28 bp, Artificial Sequence
  Description of Artificial Sequence  Synthetic Primer
tctcaggatatataaaattccttactga

(19) United States
(12) Patent Application Publication
(10) Pub. No.: US 2006/0166244 A1
(43) Pub. Date: Jul. 27, 2006

(54) DNA markers for increased milk production in cattle

(75) Inventors: Robert D. Schnabel, Columbia, MO (US); Tad S. Sonstegard, Centerville, MD (US); Curtis P. Van Tassell, Columbia, MD (US); Melissa S. Ashwell, Apex, NC (US); Jeremy F. Taylor, Columbia, MO (US)

Correspondence Address: FULBRIGHT & JAWORSKI L.L.P., 600 CONGRESS AVE., SUITE 2400, AUSTIN, TX, 78701, US

(73) Assignee: The University of Missouri System

(21) Appl. No.: 11/329,974

(22) Filed: Jan. 11, 2006

Related U.S. Application Data
(60) Provisional application No. 60/644,056, filed on Jan. 14, 2005.

(51) Int. Cl. C12Q 1/68; C07H 21/04

(52) U.S. Cl. 435/6; 536/23.2

(57) ABSTRACT

The invention provides methods for identifying a genetic polymorphism associated with altered milk production traits in dairy cattle. Genetic marker-assisted selection methods provided by the invention allow avoidance of potentially costly phenotypic testing and inaccuracies associated with traditional breeding schemes and improvement of dairy cattle herds.

This application claims benefit of and priority to U.S. Provisional Patent Application 60/644,056, filed Jan. 14, 2005, which is herein incorporated by reference in its entirety.

### BACKGROUND OF THE INVENTION

1. Field of the Invention

The present invention relates generally to the field of mammalian genetics. More particularly, it concerns genetic markers for the selection of cattle having a genetic predisposition for increased milk production traits and altered milk quality traits.

2. Description of Related Art

The genetic basis of bovine milk production is of immense significance to the dairy industry. An ability to modulate milk volumes and content has the potential to alter farming practices and to produce products which are tailored to meet a range of requirements. In particular, a method of genetically evaluating bovine to select those which express desirable traits, such as increased milk production and improved milk composition, would be desirable.

One area of success has been the identification of quantitative trait loci (QTL) associated with milk quality and quantity on chromosome 14. A non-conservative lysine to alanine substitution (K232A) in the bovine acylCoA:diacylglycerol acyltransferase (DGAT1) gene has been shown to be the causative mutation affecting variation in milk yield and composition traits of Holstein cows (Grisart et al., 2002, 2004; U.S. Patent Appl. Pub. No. 20040076977). The alanine allele produces an increase in overall milk yield and protein, but also decreases milk fat. Although the alanine allele is under positive selection in the U.S. Holstein population, in which overall milk yield has been primarily selected for, the lysine allele has been selected for in New Zealand dairy cattle populations, where increased milk fat is of primary economic importance (Spelman et al., 2002).

In addition to chromosome 14, almost all dairy cattle genome scans have identified QTL on chromosome 6. While several studies have reported a QTL affecting milk protein percent (PP) near marker BM143, some studies have indicated the presence of additional QTLs affecting various of the milk production traits suggesting either closely linked genes and/or pleiotropy. The genes and causal mutations underlying the chromosome 6 milk QTL have yet to be identified, however, several recent reports have focused upon the QTL affecting protein percentage (PP) near BM143. Ron et al., (2001) localized this QTL to a 4 cM region around BM143 (55.4 cM) in the Israeli Holstein population and identified a second QTL near marker BM415 (80.5 cM). Freyer et al., (2002) reported two QTLs for milk yield (MY) at positions 41 and 91 cM, two QTLs for PP at 44 and 67 cM, as well as a QTL affecting both fat and protein yield at 70 cM. Olsen et al., (2004) refined the position of the fat percentage (FP) and PP QTL near BM143 to a 7.5 cM interval bounded by markers BMS2508 and FBN12, which is in close agreement with the localization of Ron et al., (2001). Recently, they were able to fine map this QTL to a 420 kb interval between genes ABCG2 and LAP3. However, specific genes for this QTL have not been identified.

While the previous studies have increased the understanding of cattle genetics, there remains a need for the identification of causal polymorphisms underlying many important traits. The identification of such polymorphisms could allow implementation of accurate and inexpensive genetic assays and minimize the need for reliance on inaccurate or expensive phenotypic assays and linkage analysis studies.

### SUMMARY OF THE INVENTION

The invention relates in one aspect to the sequencing and identification of bovine osteopontin gene (OPN) polymorphisms responsible for milk production traits, for example, milk yield, milk fat percent and milk protein percent. One embodiment of the present invention provides an isolated nucleic acid molecule comprising the nucleic acid sequence of SEQ ID NO: 1 having one or more polymorphisms at a nucleic acid base positions 1406, 3379, 3490, 3492, 3907, 5075, 5896, 10043 or 11740. More specifically, the polymorphisms in one embodiment may be defined as T1406C, G3379T, G3490A, A3492G, T3907del, C5075T, G5896A, T10043C, and A11740C. Among these all but G5896A, T10043C, and Al 1740C are in the non transcribed portion of the OPN gene. While G5896A and T10043C are transcribed, they are processed from the mature mRNA and are not translated. Additionally, A11740C is transcribed but is not translated. Detection from genomic DNA will therefore be the method of choice in typical embodiments.

Still further, the present invention provides a quantitative trait nucleotide (QTN) in the upstream regulatory region of the bovine osteopontin (OPN) gene. This QTN effects milk fat percent, milk protein percent and milk yield. In particular, this QTN relates to the polymorphism in the OPN gene at position 3907 of SEQ ID NO 1. In certain embodiments, the OPN alleles characterized by the 3907 deletion produces alleles with 9 thymines and are associated with milk production traits of increased milk yield, decreased milk fat percent and decreased milk protein percent. OPN alleles not possessing a 3907 deletion produce alleles with 10 thymines and are associated with milk production traits of decreased milk yield, increased milk fat percent and increased milk protein percent. Thus, depending upon the desired milk product, it is possible to select for the appropriate allele for the desired product. For example, if a liquid dairy product is desired, then allele 3907del may be selected, and if a non-liquid dairy product is desired (e.g., cheese or butter), then allele 3907T may be selected.

Another embodiment of the invention provides a method of determining the genetic predisposition of a bovine for altered milk production traits comprising genotyping the bovine to determine the genotype for OPN. Genotyping may be carried out by assaying of genetic material from the bovine to determine the presence or absence of a polymorphism. More particularly, in one embodiment, the presence or absence of a polymorphism at position 3907 is determined.

Such a polymorphism may be detected by any method as will be understood by those of skill in the art. One convenient method for detection comprises use of the polymerase chain reaction (PCRTm). This and other techniques are well known to those of skill in the art as described herein below. Genetic material assayed is typically comprised of genomic DNA. This can be obtained from cattle post-birth, or may be obtained from fetal animals, including from embryos in vitro. The selection may comprise embryo transfer of the embryo, such that the first head of dairy cattle is grown from the embryo. The methods of the invention may be used in connection with any type of dairy cattle.

Another embodiment of the present invention comprises a method of breeding dairy cattle having altered milk production traits, comprising the steps of: (a) assaying at least one candidate head of dairy cattle to identify a first parent head of dairy cattle comprising a genetic polymorphism in OPN that confers altered milk production traits; and (b) breeding the first parent head of dairy cattle with a second parent head of dairy cattle to obtain a progeny head of dairy cattle with the polymorphism and altered milk productions trait relative to a progeny lacking the polymorphism.

In certain embodiments, the invention provides a method of obtaining a head of dairy cattle comprising a genetic predisposition for altered milk production traits, the method comprising the steps of: (a) genotyping at least a first head of dairy cattle for a genetic polymorphism in OPN associated with altered milk production traits in female dairy cattle comprising the polymorphism; and (b) selecting a head of dairy cattle having the polymorphism. In particular embodiments of the invention, the genetic polymorphism may be further defined as a deletion of a thymine at position 3907 in the bovine OPN gene. Genotyping the first parent head of dairy cattle for the presence of the genetic polymorphism in OPN may comprise, in addition to direct testing of the parent, testing of one or both of the parents of the parent to determine the genotype of the first parent.

In yet another embodiment, the invention provides a method of breeding cattle to increase the probability of obtaining progeny having a genetic predisposition for altered milk production traits, the method comprising the steps of: (a) selecting a first parent head of dairy cattle for the presence of a genetic polymorphism in OPN associated with improved or altered milk production traits in female dairy cattle comprising the polymorphism; and (b) breeding the first parent head of dairy cattle with a second parent head of dairy cattle to obtain at least a first progeny head of dairy cattle comprising the polymorphism. The method may further comprise selecting the second parent head of dairy cattle based on the genetic polymorphism in OPN. Selecting the first or second parent head of dairy cattle for the presence of the genetic polymorphism in OPN may comprise direct testing of the parent, as well as one or both of the parents of the first and/or second parent.

In one embodiment of the invention, the foregoing techniques may be used to select for OPN genotypes associated with decreased overall milk yield, for example, allele 3907T. Such a selection may be used, for example, to provide other benefits, including increased milk protein percent or fat percent. By selecting for decreased milk yield and increased protein and fat percent, this milk composition may be improved for the manufacture of dairy products that require removal of water from the milk, such as cheese and butter. The invention therefore encompasses any of the methods described herein wherein a 3907del or 3907T allele of OPN is selected.

In yet another embodiment of the invention, a method is therefore provided comprising (a) genotyping at least a first head of dairy cattle for a 3907del allele in OPN; and (b) selecting a head of dairy cattle having the polymorphism. The invention therefore also provides a method comprising the steps of: (a) selecting a first parent head of dairy cattle for the presence of a genetic polymorphism in OPN associated with increased milk fat or protein percent in female dairy cattle comprising the polymorphism; and (b) breeding the first parent head of dairy cattle with a second parent head of dairy cattle to obtain at least a first progeny head of dairy cattle comprising the polymorphism.

In a method of the invention, one or both of the first parent head of dairy cattle and the second parent head of dairy cattle may be any dairy cattle type. The method may still further be defined as comprising crossing a progeny head of dairy cattle with a third head of dairy cattle to produce a second generation progeny head of dairy cattle. The third head of dairy cattle may be a parent of the progeny head of dairy cattle or may be unrelated to the progeny head of dairy cattle. In certain embodiments of the invention, the aforementioned steps are repeated from about 2 to about 10 times, wherein the first parent head of dairy cattle is selected from a progeny head of dairy cattle resulting from a previous repetition of step (a) and step (b) and wherein the second parent head of dairy cattle is from a selected cattle breed into which one wishes to alter milk production traits. This technique will therefore allow, for example, the introduction of the beneficial characteristic into a genetic background otherwise lacking the trait but possessing other desirable traits.

The foregoing has outlined the features and technical advantages of the present invention in order that the detailed description of the invention that follows may be better nderstood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. show F-statistic profiles from the across-family analyses of segregating sire families using QTL Express (Seaton et al. 2002). Vertical bars are bootstrap replicate estimates of QTL position and are relative to the right axis. Marker locations are indicated by triangles. Horizontal lines represent chromosome-wise P<0.05 and P<0.0l critical values. FIG. 1A shows milk yield. FIG. 1B shows protein yield. FIG. 1C shows fat percentage. FIG. 1D shows protein percentage.

FIGS. 2A and 2B show joint analysis of segregating families using LDVCM (LK-linkage only, LK/LD-linkage/linkage disequilibrium) (Blott et al., 2003) and LOKI (Heath, 1997). LDVCM results are relative to the left axis which is a LOD score and LOKI results are relative to the right axis which is a Bayes factor. Marker locations are indicated by triangles. FIG. 2A shows milk percentage. FIG. 2B shows protein percentage.

### DESCRIPTION OF ILLUSTRATIVE EMBODIMENTS

Several studies have sought to identify the QTL near BM143 on chromosome 6 (BTA6) which has a large effect on milk protein percent (Ron et al., 2001, Freyer et al., 2002, Freyer et al., 2003 and Olsen et al., 2004 This 420 Kb interval contains six known human orthologs, one of which is osteopontin (OPN). The present inventors have identified polymorphisms within the OPN gene that effect milk traits. Thus, the invention provides, in one aspect, methods and compositions for the improvement of milk production in dairy cattle. The present inventors used a large multi-generation Holstein pedigree and a targeted dense marker map to map a QTL affecting milk protein percent to a relatively small interval on BTA6 in the vicinity of BM143. Examination of the genes in the region with conserved synteny on HSA4 identified Osteopontin (OPN, SPP1, Eta-1) as an ideal functional candidate gene for this QTL. OPN is a secreted glycoprotein which functions by mediating cell-matrix interactions and cellular signaling through binding with integrin and CD44 receptors and is expressed in a number of different tissues (Denhardt et al., 1993).

Sequencing of the OPN gene (SEQ ID NO 1; GenBank Accession No. AY878328) identified several polymorphisms including, for example, but not limited to T1406C, G3379T, G3490A, A3492G, T3907del, C5075T, G5896A, T10043C, and A11740C. More particularly, a polymorphism that results in altered milk yield, milk protein and fat percent is T3907del (SEQ ID NO: 1).

Another aspect of the present invention is utilizing the above listed polymorphisms as DNA markers to assist in the genotyping of the bovine by determining the presence or absence of one or more of the polymorphisms in the OPN gene. Genotyping bovine animals using the polymorphisms of the present invention, for example, T3907del, can be used to select genotypes associated with altered milk production traits, such as milk yield and milk fat and protein percent. Thus, the use of genetic assays to identify the polymorphisms identified herein as associated with altered milk production traits will find use in breeding or selection of dairy cattle produced for altered milk production traits. Thus, one embodiment of the invention comprises a breeding program directed at enhancement of milk production characteristics or traits in dairy cattle breeds adapted for milk production. In addition to herds that have increased milk yield, the milk composition from such herds, may also have altered or decreased protein and fat percents.

Likewise, the polymorphisms in the OPN gene can be used to select cows and bulls to produce a herd of cattle that lacks the OPN polymorphisms thereby generating a herd of dairy cattle that are characterized by a decreased milk yield. In addition to decreased milk yield or volume, these dairy cattle lacking the OPN polymorphism may also produce a milk composition that has an increase in protein or fat percent. Such milk compositions with an increased protein or fat percent can be used to manufacture dairy products such as cheese and butter which require the removal of water from the milk.

I. Genetic Assays And Selections

Genetic assay-assisted selections for animal breeding are important in that they allow selections to be made without the need for raising and phenotypic testing of progeny. In particular, such tests allow selections to occur among related individuals that do not necessarily exhibit the trait in question and that can be used in introgression strategies to select both for the trait to be introgressed and against undesirable background traits (Hillel et al., 1990). However, it is has been difficult to identify genetic assays for loci yielding highly heritable traits of large effect, particularly as many such traits may not be segregating and already be fixed with near optimal alleles in commercial lines. The invention overcomes this difficulty by providing such assays for alleles that are segregating in dairy cattle populations.

In accordance with the invention any assay which sorts and identifies animals based upon OPN allelic differences may be used and is specifically included within the scope of this invention. One of skill in the art will recognize that, having identified a causal polymorphism for a particular associated trait, there are an essentially infinite number of ways to genotype animals for this polymorphism. The design of such alternative tests merely represents a variation of the techniques provided herein and is thus within the scope of this invention as fully described herein. Illustrative procedures are described herein below.

Non-limiting examples of method for identifying the presence or absence of a polymorphism include single-strand conformation polymorphism (SSCP) analysis, RFLP analysis, heteroduplex analysis, denaturing gradient gel electrophoresis, temperature gradient electrophoresis, ligase chain reaction and direct sequencing of the gene. Techniques employing PCR™ detection are advantageous in that detection is more rapid, less labor intensive and requires smaller sample sizes. Primers that may be used in this regard may, for example, comprise regions of SEQ ID NO:1 and complements thereof. A PCR™ amplified portion of the OPN gene can be screened for a polymorphism, for example, with direct sequencing of the amplified region, by detection of restriction fragment length polymorphisms produced by contacting the amplified fragment with a restriction endonuclease having a cut site altered by the polymorphism, as well as by SSCP analysis of the amplified region. These techniques may also be carried out directly on genomic nucleic acids without the need for PCR™ amplification, although in some applications this may require more labor.

Once an assay format has been selected, selections may be unambiguously made based on genotypes assayed at any time after a nucleic acid sample can be collected from an individual, such as an infant animal, or even earlier in the case of testing of embryos in vitro, or testing of fetal offspring. Any source of nuclear DNA may be analyzed for scoring of genotype. In one embodiment of the invention, nucleic acids are screened that have been isolated from the blood or semen of the bovine analyzed. Generally, peripheral blood cells are conveniently used as the source of DNA. A sufficient amount of cells are obtained to provide a sufficient amount of DNA for analysis, although only a minimal sample size will be needed where scoring is by amplification of nucleic acids. The DNA can be isolated from the blood cells by standard nucleic acid isolation techniques known to those skilled in the art.

In genetic assay-assisted breeding, eggs may be collected from selected females and in vitro fertilized using semen from selected males and implanted into other females for birth. Assays may be advantageously used with both male and female cattle. Using in vitro fertilization, genetic assays may be conducted on developing embryos at the 4-8 cell stage, for example, using PCR™,and selections made accordingly. Embryos can thus be selected that are homozygous for the desired marker prior to embryo transfer.

Use of genotype-assisted selection provides more efficient and accurate results than traditional methods. This also allows rapid introduction into or elimination from a particular genetic background of the specific trait or traits associated with the identified genetic marker. In the instant case, screening for OPN alleles conferring altered milk traits, e.g., increased milk volume and/or decreased protein and fat concentrations or decreased milk volume and/or increased protein and fat concentrations, may be used to allow the efficient culling of altered milk trait genotypes from breeding stock, as well as the introduction of non-altered milk trait genotypes into genetic backgrounds lacking the trait, as desired.

Genetic assays can be used to obtain information about the genes that influence an important trait, thus facilitating breeding efforts. Factors considered in developing markers for a particular trait include: how many genes influence a trait, where the genes are located on the chromosomes (e.g., near which genetic markers), how much each locus affects the trait, whether the number of copies has an effect (gene dosage), pleiotropy, environmental sensitivity and epistatis.

A genetic map represents the relative order of genetic markers, and their relative distances from one another, along each chromosome of an organism. During sexual reproduction in higher organisms, the two copies of each chromosome pair align themselves closely with one another. Genetic markers that lie close to one another on the chromosome are seldom recombined, and thus are usually found together in the same progeny individuals. Markers that lie close together show a small percent recombination, and are said to be linked. Markers linked to loci having phenotypic effects are particularly important in that they may be used for selection of individuals having the desired trait.

The identity of a given allele can therefore be determined by identifying nearby genetic markers that are usually co-transmitted with the gene from parent to progeny. This principle applies both to genes with large effects on phenotype (simply inherited traits) and genes with small effects on phenotype. As such, by identifying a marker linked to a particular trait, this will allow direct selection for the linked polymorphism without the need for detecting that particular polymorphism due to genetic linkage between the traits. Those of skill in the art will therefore understand that when genetic assays for OPN are mentioned herein this specifically encompasses detection of genetically linked polymorphisms that are informative for the OPN alleles. Such polymorphisms have predictive power relative to the trait to the extent that they also are linked to the contributing locus for the trait. Such markers thus also have predictive potential for the trait of interest.

Most natural populations of animals are genetically quite different from the classical linkage mapping populations. While linkage mapping populations are commonly derived from two-generation crosses between two parents, many natural populations are derived from multi-generation matings between an assortment of different parents, resulting in a massive reshuffling of genes. Individuals in such populations carry a complex mosaic of genes, derived from a number of different founders of the population. Gene frequencies in the population as a whole may be modified by natural or artificial selection, or by genetic drift (e.g., chance) in small populations. Given such a complex population with superior average expression of a trait, a breeder might wish to: (1) maintain or improve the expression of the trait of interest, while maintaining desirable levels of other traits; and (2) maintain sufficient genetic diversity that rare desirable alleles influencing the trait(s) of interest are not lost before their frequency can be altered by selection.

Genetic assays may find particular utility in maintaining sufficient genetic diversity in a population while maintaining favorable alleles. For example, one might select a fraction of the population based on favorable phenotype (perhaps for several traits—one might readily employ index selection), then apply genetic assays as described herein to this fraction and keep a subset which represent much of the allelic diversity within the population. Strategies for extracting a maximum of desirable phenotypic variation from complex populations remain an important area of breeding strategy. An integrated approach, merging classical phenotypic selection with a genetic marker-based analysis, may aid in extracting valuable genes from heterogeneous populations.

The techniques of the present invention may potentially be used with any bovine, including Bos taurus and Bos indicus. In particular embodiments of the invention, the techniques described herein are specifically applied for the selection of dairy cattle, as the genetic assays described herein will find utility in maximizing production of animal products, such as dairy products. As used herein, the term “dairy cattle” refers to cattle grown or bred primarily for the production of dairy animal products. Therefore, a “head of dairy cattle” refers to at least a first bovine animal grown or bred for production of dairy animal products. Examples of breeds of cattle that may be used with the invention include, but are not limited to, Ayrshire, Brown Swiss, Guernsey, Holstein, Jersey, Norwegian Red, Milking Devon, Kerry, Dutch Belted, Canadiene, Milking Shorthorn, Danish Jersey, Normandy, Montbeliarde, Danish Red, and British Friesian, as well as animals bred therefrom and related thereto.

II. OPN Nucleic Acids

Certain embodiments of the present invention concern OPN nucleic acid molecules encoding an isolated nucleic acid sequence that is a “wild-type” or “consensus” sequence of OPN, for example, SEQ ID NO 1 (GenBank Accession No. AY878328). More particularly, other OPN nucleic acid molecules include molecules containing polymorphisms. Examples of such polymorphisms include, but are not limited to T1406C, G3379T, G3490A, A3492G, T3907del, C5075T, G5896A, T10043C, and A11740C. In certain embodiments the polymorphism is T3907del.

The term “nucleic acid” generally refers to at least one molecule or strand of DNA or a derivative or mimic thereof, comprising at least one nucleotide base, such as, for example, a naturally occurring purine or pyrimidine base found in DNA (e.g., adenine “A”, guanine “G”, thymine “T”, and cytosine “C”). The term “nucleic acid” encompasses the terms “oligonucleotide” and “polynucleotide”. These definitions generally refer to at least one single-stranded molecule, but in specific embodiments will also encompass at least one additional strand that is partially, substantially or fully complementary to the single-stranded molecule. Thus, a nucleic acid may encompass at least one double-stranded molecule or at least one single-stranded molecule that comprises one or more complementary strand(s) or “complement(s)” of a particular sequence comprising a strand of the molecule. An “isolated nucleic acid” as contemplated in the present invention may comprise transcribed nucleic acid(s), regulatory sequences, coding sequences, or the like, isolated substantially away from other such sequences, such as other naturally occurring nucleic acid molecules, regulatory sequences, polypeptide or peptide encoding sequences, etc.

III. Nucleic Acid Detection

Techniques for nucleic acid detection may find use in certain embodiments of the invention. For example, such techniques may find use in scoring individuals for genotypes or in the development of novel markers linked to the major effect locus identified herein.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production. The invention therefore specifically provides such probes or primers that correspond to or are a complement of SEQ ID NO:1.

Accordingly, nucleotide sequences may be used in accordance with the invention for their ability to selectively form duplex molecules with complementary stretches of DNAs or to provide primers for amplification of DNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes. It is generally appreciated that conditions can be rendered more stringent by the addition of increasing amounts of formamide.

For certain applications, lower stringency conditions may be preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences with the present invention in combination with an appropriate means, such as a label, for determining hybridization. For example, such techniques may be used for scoring of RFLP marker genotype. A wide variety of appropriate indicator means are known in the art, including fluorescent, radioactive, enzymatic or other ligands, such as avidin/biotin, which are capable of being detected. In certain embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that probes or primers will be useful as reagents in solution hybridization, as in PCR™,for detection of nucleic acids, as well as in embodiments employing a solid phase. In embodiments involving a solid phase, the test DNA is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 1989). Such embodiments may find particular use with the invention, for example, in the detection of repeat length polymorphisms, such as microsatellite markers. In certain embodiments of the invention, amplification analysis is performed on whole cell or tissue homogenates or biological fluid samples without substantial purification of the template nucleic acid.

The term “primer”, as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles”, are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology). Typically, scoring of repeat length polymorphisms will be done based on the size of the resulting amplification product.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, each of which is incorporated herein by reference in their entirety.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, also may be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

An isothermal amplification method, in which restriction endonucleases and ligases are used to achieve the amplification of target molecules that contain nucleotide 5′-[alphathio]-triphosphates in one strand of a restriction site also may be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids also may be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect polymorphisms in genomic nucleic acids. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Kits

All the essential materials and/or reagents required for screening cattle for genetic marker genotype in accordance with the invention may be assembled together in a kit. This generally will comprise a probe or primers designed to hybridize specifically to individual nucleic acids of interest in the practice of the present invention, for example, primer sequences such as those for amplifying OPN. Also included may be enzymes suitable for amplifying nucleic acids, including various polymerases (reverse transcriptase, Taq, etc.), deoxynucleotides and buffers to provide the necessary reaction mixture for amplification. Such kits also may include enzymes and other reagents suitable for detection of specific nucleic acids or amplification products. Such kits generally will comprise, in suitable means, distinct containers for each individual reagent or enzyme as well as for each probe or primer pair.

In certain embodiments, the invention also can provide for a kit which can be used to determine the OPN genotype of bovine genetic material, for example the kit may include a set of primers used for amplifying the genetic material. A kit can contain a primer including a nucleotide sequence for amplifying a region of the genetic material containing one of the polymorphisms described herein. Such a kit could also include a primer for amplifying the corresponding region of the normal OPN gene, i.e., the sequence without polymorphisms. Usually, such a kit would also include another primer upstream or downstream of the region of interest complementary to a coding and/or non-coding portion of the gene. These primers are used to amplify the segment containing the mutation, i.e. polymorphism, of interest. Examples of such primers include, but are not limited to SEQ ID NO:2 and SEQ ID NO:3.

IV. Definitions

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more”, “at least one”, and “one or more than one”. Still further, the terms “having”, “including”, “containing”, and “comprising” are interchangeable and one of skill in the art is cognizant that these terms are open ended terms.

As used herein, the term “gene” is defined as a functional protein, polypeptide, peptide-encoding unit, as well as non-transcribed DNA sequences involved in the regulation of expression. As will be understood by those in the art, this functional term includes genomic sequences, cDNA sequences, and smaller engineered gene segments that express, or is adapted to express, proteins, polypeptides, domains, peptides, fusion proteins, and mutants.

As used herein, the term “genotype” or “genotypic” refers to the genetic constitution of a subject, for example, the alleles present at one or more specific loci.

As used herein, the term “genotyping” refers to the process that is used to determine the subject's genotype.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides”. The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™,and the like, and by synthetic means.

As used herein, the term “polymorphism” refers to the presence in a population of two (or more) allelic variants. Such allelic variants include sequence variation in a single base, for example a single nucleotide polymorphism (SNP).

As used herein, the term “single nucleotide polymorphisms” or “SNP” or “SNPs”, as used herein, refers to common DNA sequence variations among subjects. The DNA sequence variation is typically a single base change or point mutation resulting in genetic variation between individuals. The single base change can be an insertion or deletion of a base.

As used herein, the term “3907del” or “OPN3907del” refers to the deletion of the “thymine” base or “T” at the position in the OPN gene corresponding to position 3907 of SEQ ID NO:1. This deletion produces an allele of 9 thymines. As used herein, the term “3907T” refers to an allele that produces 10 thymines, which includes a “thymine” base or “T” at the same position.

V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Animals And Traits

DNA samples from Holstein artificial insemination sires were obtained from the Cooperative Dairy DNA Repository (CDDR) for 45 half-sib families (Ashwell and Van Tassell, 1999). Each of these half-sib families belongs to one of three extended super-families denoted as families L, M and N. The number of animals that were genotyped in each of the families is shown in Table 1. Sire identifiers consist of super-family letter (M-N), generation number (I-V) and individual identifier within generation, similar to standard pedigree nomenclature. Super-families L and N comprise 3 generations of extended half-sib families while super-family M contains 5 generations of half-sib families. All three of the founding sires (L-0, M-I-1 and N-0) and all intermediary sires that link the analyzed half-sib families to the founding sires were genotyped.

TABLE 1
Numbers of animals genotyped by family. Families are identified
by super-family code (L, M or N), generation number (I-V) and sire
ID within generation (1-23).

| ID | Sire | Number sons |
|---|---|---|
| L-I-1 | L-0 | 93 |
| L-II-3 | L-I-1 | 100 |
| L-II-4 | L-I-1 | 77 |
| L-II-5 | L-I-1 | 11 |
| L-II-6 | L-I-1 | 38 |
| L-II-7 | L-I-1 | 68 |
| L-II-9 | L-I-1 | 98 |
| L-II-10 | L-I-1 | 33 |
| L-II-11 | L-I-1 | 18 |
| L-II-14 | L-I-1 | 61 |
| L-II-15 | L-I-1 | 20 |
| L-II-16 | L-I-1 | 66 |
| L-II-17 | L-I-1 | 49 |
| L-III-3 | L-II-4 | 74 |
| L-III-13 | L-II-17 | 34 |
| M-II-1 | M-I-1 | 226 |
| M-II-7 | M-I-1 | 98 |
| M-III-9 | M-II-6 | 27 |
| M-III-10 | M-II-6 | 173 |
| M-III-11 | M-II-7 | 46 |
| M-III-12 | M-II-7 | 12 |
| M-III-13 | M-II-7 | 25 |
| M-III-15 | M-II-7 | 20 |
| M-III-16 | M-II-7 | 49 |
| M-III-17 | M-II-7 | 126 |
| M-III-18 | M-II-7 | 114 |
| M-III-19 | M-II-7 | 83 |
| M-III-22 | M-II-7 | 12 |
| M-III-23 | M-II-7 | 158 |
| M-IV-6 | M-III-10 | 94 |
| M-IV-8 | M-III-10 | 64 |
| M-IV-16 | M-III-19 | 28 |
| M-V-14 | M-IV-8 | 194 |
| N-I-1 | N-0 | 167 |
| N-II-1 | N-I-1 | 63 |
| N-II-4 | N-I-1 | 139 |
| N-II-2 | N-I-1 | 17 |
| N-II-6 | N-I-1 | 84 |
| N-II-5 | N-I-1 | 49 |
| N-III-1 | N-II-4 | 109 |
| N-III-2 | N-II-4 | 15 |
| N-III-3 | N-II-4 | 54 |
| N-III-4 | N-II-4 | 21 |
| N-III-5 | N-II-6 | 40 |

### EXAMPLE 2

### Genotyping

Microsatellite markers (N=38; Table 2) were chosen from public databases (www.marc.usda.gov) and the forward primer of each marker was synthesized with one of 3 fluorescent labels (6-FAM, HEX or NED). Multiplex reactions were developed based on the allele size ranges, fluorescent label and the ability of each marker to co-amplify. Between 4 and 8 markers were co-amplified in each reaction. PCR™ was performed using 5 μl reactions on an ABI 9700 thermocycler (Applied Biosystems) using protocols based on Schnabel et al., (2004). PCR™ products were separated on an ABI 3700 Automated Sequencer and sized relative to the GS400HD internal size standard (Applied Biosystems). Fluorescent signals from the dye labeled microsatellites were detected using GENESCAN 3.1 (Applied Biosystems) and genotypes were assigned using Genotyper 3.7 (Applied Biosystems). Not all families were genotyped for every marker because initial genotyping focused only on markers in which the sire was informative. All families were genotyped for the DGAT1 K232A mutation (Grisart et al., 2002).

### EXAMPLE 3

### QTL Express

Each family was analyzed individually under a grand-daughter design model using QTL Express (Seaton et al., 2002) to determine the segregation status of each sire for BTA6 QTL for each trait. Data permutation (5000 replicates) was used to determine chromosome-wise significance levels for each sire (Churchill and Doerge 1994). Tests of one vs. zero, one vs. two and two vs. zero QTL were conducted individually for each sire family. Sires that were significant at the chromosome-wise P<0.05 level for the one QTL model were classified as segregating, regardless of trait or QTL position. All segregating sires were combined into a “segregating” dataset. Additionally, sires that were significant for the two-QTL model or demonstrated evidence of two QTL were also added to the segregating dataset which included 22 families. Across family analysis was then performed on the segregating dataset. Bootstrapping (1,000 replicates) was performed to estimate QTL location across families (Visscher et al., 1996). Determination of significance levels using data permutation is not an option using the two-QTL model of QTL Express due to computational limitations. Therefore, to account for multiple testing in the two-QTL models we used the following approach. For the one-QTL model, F-statistics were generated based on data permutation to represent the chromosome-wise P<0.05 and P<0.01 levels. For example, the chromosome-wise P<0.05 level based on data permutation for a sire with 93 sons required an F-statistic of 6.33. The exact P-value corresponding to F=6.33 as an observation on an F distribution with 1 numerator and 92 denominator df is P=0.0136. Thus, in order for the two-QTL model to be considered significant at the P<0.05 level, the uncorrected P-value associated with the two-QTL F-statistic must be less than P=0.0136. Sires that were significant for the one-QTL model were then evaluated for the two vs. one model and sires that were not significant for the one-QTL model were evaluated for the two vs. zero QTL model.

### EXAMPLE 4

### LOKI

To limit computational complexity for the across family analyses, LOKI v2.4.5 (Heath 1997) was used for multipoint QTL analysis using the dataset for the segregating sire families. LOKI was also used to analyze each half-sib family individually to estimate both the number and position of QTL for each sire. An initial burn in of 1,000 iterations was followed by 501,000 iterations where parameter estimates were collected at every iterate for a total of 500,000 data points.

A description of the analytical model and the MCMC sampling process is presented in Heath (1997). Briefly, the trait is modeled by k biallelic QTL where for the i^{th} QTL, genotypes A₁A₁, A₁A₂, and A₂A₂ have genotypic effects aᵢ, dᵢ and −aᵢ, respectively. The model for trait y (n×1; n animals each with a single observation) can be expressed as:

$$y = μ + X β + {\sum }_{i = 1}^{k} {Q}_{i} {α}_{i} + Zu + e$$

where: μ is the overall trait mean, β is an (m×1) vector of fixed effects and covariates, αᵢ, is a (2×1) vector of allele substitution effects for the i^{th} QTL, u is an (n×1) vector of random normally distributed additive residual polygene effects, e is an (n×1) vector of normally distributed residuals, k is the number of QTL in the model and X (n×m), Qᵢ (n×2) and Z (n×n) are known incidence matrices for the fixed, QTL and polygenic effects, respectively. DGAT1 genotypes were included in the model as a fixed effect. LOKI offers the analytical advantage of allowing the number of QTL in the model to vary while simultaneously analyzing the entire genome. In this case, since only one chromosome was genotyped, the total genome length was set to 2,900 cM to fit additional unlinked QTL.

### EXAMPLE 3

### Mapping

A linkage map for BTA6 was constructed using CRI-MAP v. 2.4 (Green et al., 1990). The BUILD option was used to construct a framework map of markers for which support for locus order was LOD≧3. The remaining markers were incorporated into the map in order according to their number of informative meioses using the ALL option. The FLIPS option was used to evaluate the support for local permutations of marker order. Finally, the CHROMPIC option was used to identify spurious double recombinants and to facilitate the correction of genotyping errors.

Genoprob (Thallman et al., 2001 a,b) was also used to quality assure genotype scores. All genotyped individuals and their non-genotyped mothers were assembled into a single pedigree to exploit the full pedigree structure of the U.S. Holstein population. Genotype and grand-parental origin probabilities for each marker genotype were estimated for each of the animals in this pedigree based on all available information (genotypes, genetic map and pedigree). Only genotypes that had genotype probabilities ≧0.95 (as defined in Genoprob) were included in the QTL analyses.

In order to integrate the bovine linkage and human physical maps, two methods were used to map bovine microsatellites to the human sequence. First, bovine BAC clones harboring the markers BMS2508 and BMS5015 were identified by screening high density filters using overgo oligonucleotide hybridization. These two markers were selected due to their likelihood of flanking the QTL. Positive BAC clones were subcloned, shotgun sequenced and the sequences queried against the human sequence assembly (//genome.ucsc.edu/) using BLAT. Second, the sequence of each microsatellite marker genotyped on BTA6 was queried against the Bos taurus trace archive (www.ncbi.nih.gov/Traces/) using BLAST.

The sex-specific, Holstein BTA6 linkage map is presented in Table 2. Due to the computational limitations associated with such a large number of markers and meioses it was not possible to perform a full BUILD of the map. Therefore, a LOD≧3 framework map was first constructed using the most informative markers, less informative markers were inserted into the map using the ALL option and local marker order was tested using a sliding window of 5-10 markers and the FLIPS option of Cri-Map. The marker order agrees with the previously reported maps, which were based on many fewer informative meioses per marker, except for markers separated by sub-centimorgan distances. By aligning the bovine microsatellites to the human genome sequence (Table 2) it appeared that the linkage-assigned order for markers BM3026 and BMS483 was not correct. However, given the close proximity (<500 kb) of these markers and the number of closely-linked flanking markers, it was found that changing the order of these markers had no appreciable affect on the QTL analyses.

TABLE 2
BTA6 linkage map and marker positions relative to the USDA linkage
map and human chromosome 4 physical map.

| | USDA | HSA4 | Haldane | Number | Number of |
|---|---|---|---|---|---|
| | position | position | position | of informative | families |
| Marker | (cM)¹ | (Mb)² | (cM) | meioses | genotyped |
| ILSTS093 | 0.00 | NA | 0.0 | 2021 | 42 |
| INRA133 | 8.05 | 116.14 | 18.0 | 772 | 40 |
| BMS5006 | 17.00 | 112.24 | 23.6 | 2563 | 40 |
| URB016 | 34.45 | NA | 46.1 | 2314 | 40 |
| BMS2508 | 43.94 | 93.58 | 53.9 | 1982 | 42 |
| MNB175* | 47.29 | 18.52 | 56.8 | 1207 | 42 |
| BMS5037 | 47.82 | 18.97 | 56.9 | 2678 | 40 |
| BMS382 | 51.43 | 21.10 | 58.5 | 1979 | 40 |
| FBN12 | NA | 22.32 | 59.5 | 1548 | 40 |
| BM3026 | 52.78 | 23.11 | 59.8 | 2206 | 40 |
| BMS1242* | 52.84 | 22.43 | 59.9 | 2335 | 40 |
| MNB203* | 52.78 | 22.66 | 60.0 | 1534 | 40 |
| BM143 | 53.72 | 22.89 | 60.1 | 2813 | 40 |
| MNB196 | 57.00 | NA | 61.2 | 973 | 40 |
| BMS5015 | 56.44 | 25.12 | 63.4 | 2411 | 40 |
| BMS690* | 56.44 | 25.46 | 63.5 | 2351 | 42 |
| DIK082 | 57.57 | NA | 64.2 | 3376 | 42 |
| MNB192 | 58.97 | 31.40 | 66.0 | 2030 | 42 |
| TGLA37* | 59.74 | NA | 66.1 | 1205 | 42 |
| BMS518* | 58.97 | NA | 66.5 | 976 | 40 |
| BMS5010 | 61.70 | 33.00 | 66.6 | 2826 | 31 |
| BMS5033* | 67.82 | NA | 69.1 | 958 | 39 |
| BMS483* | 67.82 | 37.70 | 69.3 | 1294 | 37 |
| BMS470 | 67.40 | 37.31 | 69.4 | 1433 | 26 |
| BMS360 | 72.88 | 43.76 | 72.6 | 2764 | 29 |
| CA028 | 79.19 | NA | 78.4 | 1999 | 31 |
| BMS5028 | 81.96 | NA | 80.5 | 508 | 42 |
| BM415* | 81.96 | 57.92 | 80.6 | 1336 | 17 |
| BMS5032 | 81.96 | 58.33 | 80.7 | 1584 | 42 |
| MB062 | 89.34 | 71.28 | 89.3 | 872 | 22 |
| BM1236 | 90.51 | 73.35 | 91.3 | 1570 | 13 |
| BMS2460 | 93.45 | NA | 92.7 | 1875 | 40 |
| BMS5021* | 93.85 | NA | 92.8 | 676 | 42 |
| BM4311 | 97.73 | NA | 93.4 | 1418 | 30 |
| BP7 | 98.50 | NA | 94.0 | 1105 | 42 |
| BM8124 | 101.41 | 81.48 | 96.6 | 633 | 18 |
| BMS5029 | 118.08 | 10.19 | 113.4 | 2278 | 26 |
| BMC4203* | 119.05 | 10.37 | 113.8 | 1315 | 15 |

¹Ihara et al., (2004)
²May 2004 assembly (http://genome.ucsc.edu)
*Support for marker order less than LOD 3.0

### EXAMPLE 4

### QTL Analysis

Milk production phenotypes, daughter yield deviations (DYD) and predicted transmitting abilities (PTA), were obtained from the Animal Improvement Programs Laboratory of the USDA (May 2004 evaluations). Traits analyzed were milk, fat and protein yield (MY, FY and PY) as well as the percentage traits, fat and protein percent (FP and PP). Three distinct approaches were used for QTL analysis; 1. Half-sib least squares regression using QTL Express (Seaton et al., 2002), 2. Full pedigree MCMC analysis using LOKI (Heath 1997) and 3. Combined linkage/linkage disequilibrium analysis using LDVCM (Blott et al., 2003). All QTL analyses used the male specific genetic map with marker locations in Haldane centimorgans (Table 2).

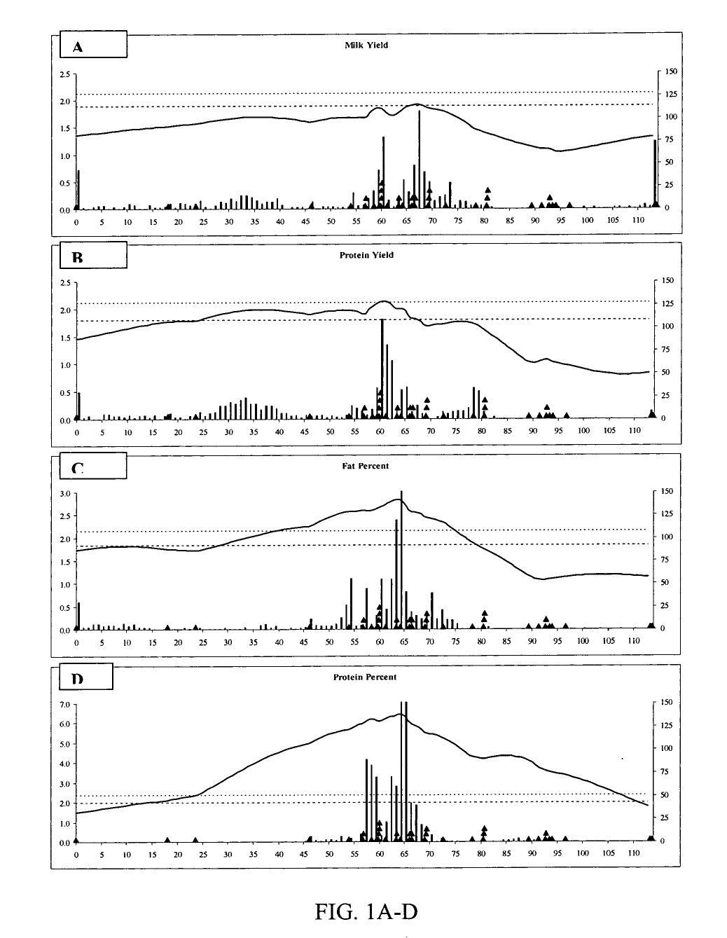

A total of 3,147 individuals from 45 families (mean=72) were available for QTL analysis. Twenty six sires representing all three super-families were determined to be segregating for at least one of the 5 milk production phenotypes based on the within-family analyses (Table 3). Eleven sires were statistically significant for the two-QTL model. The across family F-statistic profiles based on these 26 sires are shown in FIG. 1. Results for fat yield were not significant at a chromosome-wise P<0.05 in the across-family analysis. Peak test statistics in the across-family analysis were: MY at 59cM and 67cM, FP at 64cM, PY at 61cM and PP at 64cM. Since there are multiple QTL influencing milk traits on BTA6, the test statistic profiles for the single QTL model analyses in FIG. 1 were not informative for the number of segregating QTL or their positions. Similarly, the use of the bootstrap to estimate confidence intervals for QTL location assumed a single segregating QTL, however, an examination of the distribution of the bootstrap replicates revealed clusters corresponding to locations that were consistent between traits; 0cM (MY, FP & PY), 59-61cM (MY, PY & PP), 64-68cM (MY, FP & PP) and 113cM for MY only. The localization of QTL to these regions was supported by the individual family analyses in which sires were identified as segregating for QTL at all of these locations (Table 3).

TABLE 3
Results of QTL analysis.

| | One-QTL Model | | | Two-QTL Model^{†} | | | | LOKI^{†} | | | |
|---|---|---|---|---|---|---|---|---|---|---|---|
| Sire | Trait | F | Loc | F | Loc 1 | Loc 2 | Markers | BF 1 | Loc 1 | BF 2 | Loc 2 |
| L-I-1 | MY | 8.59* | 109 | | | | | 70.1 | 106 | | |
| | FP | 26.83** | 57 | | | | | 336.0 | 57 | | |
| | PY | 5.35 | 60 | 5.57* | 60 | 113 | 18 | 0.9 | 63 | | |
| | PP | 66.71** | 57 | | | | | 394.3 | 58 | | |
| L-II-4 | PP | 6.78* | 57 | | | | | 4.0 | 63 | 3.8 | 58 |
| L-II-14 | FY | 10.25** | 64 | | | | | 1.5 | 63 | | |
| | FP | 21.85** | 45 | | | | | 52.2 | 63 | | |
| | PP | 21.08** | 52 | | | | | 65.6 | 51 | | |
| L-II-15 | FP | 14.52** | 71 | | | | | 1.8 | 81 | | |
| | PP | 18.27** | 52 | | | | | 3.5 | 45 | | |
| L-II-16 | MY | 6.66* | 60 | | | | | 3.2 | 49 | | |
| | PY | 7.80* | 7 | | | | | 2.3 | 21 | | |
| L-II-17 | PP | 4.29 | 76 | 5.03* | 59 | 64 | | 1.5 | 4 | | |
| M-II-1 | MY | 3.15 | 78 | | | | | 248.7 | 93 | | |
| | FY | 9.67* | 69 | | | | | 2.1 | 92 | | |
| | FP | 5.14 | 25 | | | | | 14.6 | 93 | | |
| M-II-7 | FY | 2.74 | 0 | 5.96* | 82 | 97 | 5 | 1.4 | 69 | | |
| | FP | 6.75* | 0 | | | | | 4.6 | 7 | | |
| M-II-6 | PP | 13.62** | 65 | | | | | 66.3 | 66 | | |
| M-III-10 | MY | 6.12 | 24 | | | | | 11.6 | 2 | | |
| | PY | 15.79** | 34 | | | | | 9.8 | 65 | 6.8 | 56 |
| | PP | 6.64 | 46 | | | | | 20.9 | 56 | 18.2 | 67 |
| M-III-11 | FY | 3.20 | 40 | 5.13* | 61 | 81 | | 3.3 | 17 | | |
| | PY | 3.12 | 32 | 6.77* | 46 | 78 | | 21.8 | 68 | | |
| M-III-12 | PY | 32.19** | 101 | | | | | 1.4 | 94 | | |
| M-III-13 | FP | 0.36 | 0 | 6.08* | 93 | 106 | 2 | 1.7 | 71 | | |
| M-III-16 | MY | 2.19 | 94 | | | | | 28.1 | 18 | | |
| | PP | 3.40 | 94 | | | | | 17.5 | 66 | | |
| M-III-19 | MY | 3.44 | 102 | 5.88* | 71 | 83 | 2 | 0 | 0 | | |
| M-III-23 | PP | 1.02 | 81 | | | | | 12.9 | 46 | 12.5 | 25 |
| M-IV-6 | PY | 6.44 | 59 | 5.62* | 59 | 92 | | 3.4 | 114 | | |
| | PP | 10.42* | 73 | | | | | 48.8 | 65 | 37.0 | |
| M-IV-8 | FY | 6.86* | 0 | | | | | 11.5 | 25 | | |
| | PY | 6.05 | 4 | | | | | 37.3 | 114 | | |
| N-I-1 | FP | 1.22 | 5 | | | | | 10.1 | 60 | | |
| | PP | 11.29** | 89 | | | | | 22.5 | 90 | | |
| N-II-1 | FP | 8.51* | 70 | | | | | 3.5 | 60 | | |
| | PP | 4.85 | 91 | | | | | 55.0 | 63 | | |
| N-II-4 | PY | 3.41 | 65 | | | | | 12.7 | 114 | | |
| | PP | 28.33** | 87 | | | | | 122.4 | 90 | | |
| N-II-2 | FY | 4.48 | 58 | 7.49* | 71 | 91 | | 1.7 | 69 | | |
| | PY | 3.37 | 1 | 6.44* | 64 | 70 | | 1.3 | 114 | | |
| N-II-5 | FP | 6.26 | 64 | 9.14** | 68 | 79 | | 1.0 | 88 | | |
| | PP | 10.54* | 64 | | | | | 2.7 | 64 | | |
| N-II-6 | PP | 8.17* | 5 | | | | | 6.3 | 68 | | |
| N-III-2 | MY | 8.13* | 69 | 10.04* | 70 | 97 | 3 | 2.5 | 51 | | |
| | FP | 4.01 | 105 | 9.42* | 72 | 93 | 2 | 1.7 | 66.5 | | |
| | PP | 3.02 | 69 | 11.47** | 65 | 90 | 6 | 1.9 | 46.5 | | |
| N-III-3 | FY | 11.01** | 113 | 9.78** | 68 | 102 | 7 | 12.4 | 66.5 | | |
| | FP | 2.19 | 112 | | | | | 11.3 | 1.5 | | |
| | PY | 12.2** | 104 | | | | | 2.7 | 50.5 | | |

*Chromosome-wide P < 0.05
**Chromosome-wide P < 0.01
^{†}Loc 1 and Loc 2 refer to the two peak test statistics from the two-QTL model. BF 1 and BF 2 refer to the Bayes factor associated with Loc 1 and Loc 2 for the LOKI analysis

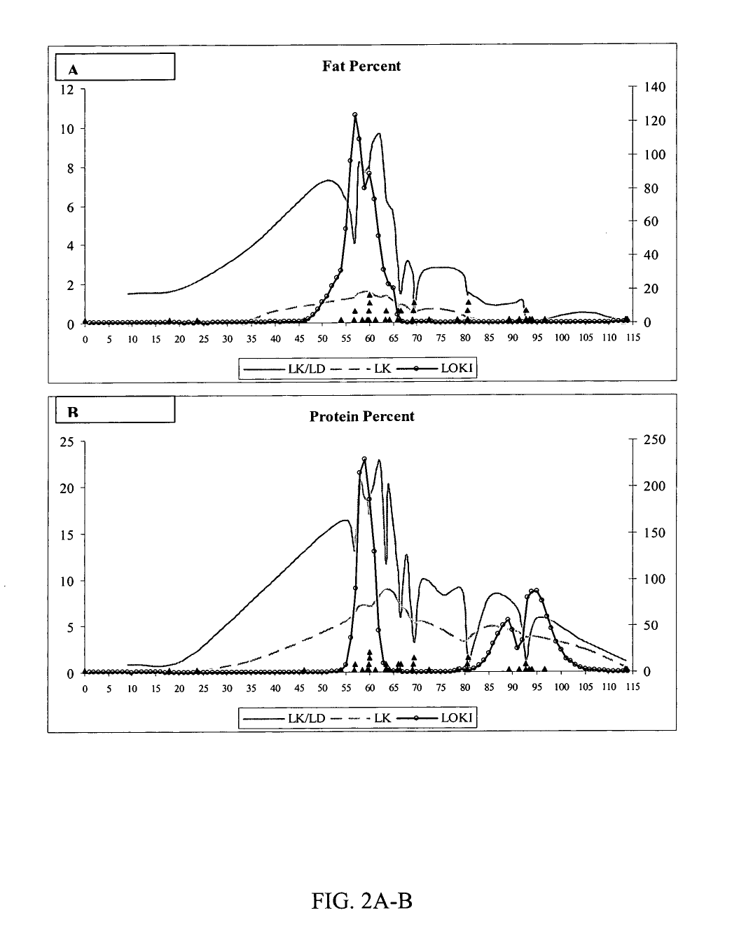

The across-family results for FP and PP using LOKI and LDVCM are shown in FIG. 2. LOKI indicated the presence of two QTL for FP at 57 cM (Bayes Factor (BF)=123) and 60 cM (BF=88) and three QTL for PP at 59 cM (BF=229), 89 cM (BF=56) and 95 cM (BF=86). The 95% highest posterior density interval for the PP peak at 57 cM was 7.2 cM (55.0 cM-62.2 cM) which included 60 cM. LDVCM indicated the presence of two QTL for FP at 57 cM (LOD=8.2) and 62 cM (LOD=9.6) and six QTL for PP at 57 cM (LOD=20.5), 62 cM (LOD=22.5), 64 cM (LOD=20.2), 68 cM (LOD=12.7), 85 cM (LOD=8.0) and 95 cM (LOD=5.9). Both LOKI and LDVCM provided evidence for QTL at many of the same positions as identified by the bootstrap analysis from QTL Express. However, it was also very clear that both LOKI and LDVCM were able to resolve these QTL in an across-family analysis.

The linkage disequilibrium analysis produced several distinct peaks suggesting the presence of a number of QTL, including three within a 7 cM interval for PP To test if marker information content affected the number of detected QTL all markers with <1,000 informative meioses (Table 2) were removed and reanalyzed by LDVCM. The resulting LOD profiles were identical to those in FIG. 2 indicating that these peaks were not artifactual due to marker information content. Considering that 3,147 animals were genotyped for 14 markers between 56.0 and 66.1 cM, these results strongly suggested the presence of three QTL in this region. Additionally, both the LOKI and LDVCM analyses indicated a PP QTL near the casein cluster (90 cM) and another positioned near 95 cM. The locations of both of these QTL were consistent with those estimated from the within-family analyses (Table 3).

### EXAMPLE 5

### Sequencing OPN

The osteopontin (OPN) gene was identified as a strong functional candidate gene for the QTL affecting PP located at 57cM.

PCR™ primers were developed within the exons of OPN based on the TIGR consensus sequence TC152671 which has subsequently been replaced by TC26249 (www.tigr.org). After the introns were sequenced, primers were designed within flanking introns to sequence each exon. In order to sequence the 5′ and 3′ regions of the gene, BAC 263K19 was identified from the CHORI-240 bovine BAC library using overgo hybridization. Sequencing primers were used to obtain approximately 5,000 bp of sequence upstream of the transcription initiation site and 200 bp past the poly-A signal -from this clone. From this sequence, PCR™ primers were developed to allow the complete sequencing of OPN and flanking regions in individual animals. A total of 8 sires were sequenced for the entire 12.3 kb region harboring OPN; four sires identified as segregating (Qq) for the QTL within the 420 kb critical region, and four non-segregating sires (QQ or qq) (See Genbank accession number AY878328).

In these 8 sires, a total of 9 SNPs were found. SNP locations were numbered according to position within the consensus sequence in AY878328 and the detected SNP haplotypes are presented in Table 4. The four segregating sires that were chosen for sequencing all shared the PP decreasing QTL allele identical by descent (IBD). Thus, a single SNP was responsible for the detected variation in PP and in order for any of the detected SNPs to be a QTN candidate, the SNP genotypes was concordant with the QTL genotypes of all 4 segregating (heterozygous) and 4 non-segregating (homozygous) sires.

Table 4 revealed that the only concordant SNP was T3907del which was an indel located approximately 1,240bp upstream of the OPN transcription initiation site. The T3907del indel occurred within a poly-T tract producing alleles of either 9 or 10 thymines. Primers were designed using a fluorescently labeled forward primer to genotype this SNP as a fragment length polymorphism (SEQ ID NO 2: OPN3907F: 5′-tccataattttctttcaaacacctt-3′ and SEQ ID NO 3: OPN3907R 5′-tctcaggatatataaaattccttactga-3′). The G3379T, G3490A and A3492G SNPs were also genotyped by allele-specific PCR™ using a modification of the procedure of Drenkard et al., (2000). All 4 SNPs were genotyped in a panel of 167 sires that represent all of the sire lines contained in the CDDR.

TABLE 4
OPN SNP haplotypes detected in eight sequenced sires. Segregation status for
the QTL located near BM143 is indicated by assigning QTL alleles Q or q to each
haplotype. SNPs are numbered according to base position in the consensus sequence in
accession number AY878328. For each sire, the first row represents the maternally
inherited and the second row the paternally inherited haplotype.

| Sire | QTL | T1406C | G3379T | G3490A | A3492G | T3907del | C5075T | G5896A | T10043C | A11740C |
|---|---|---|---|---|---|---|---|---|---|---|
| L-I-1 | Q | T | G | G | G | 3907del | C | G | T | C |
| | q | T | T | G | A | 3907T | C | G | C | A |
| L-II-14 | Q | T | G | G | G | 3907del | C | G | T | C |
| | q | T | T | G | A | 3907T | C | G | C | A |
| L-II-15 | Q | T | G | G | G | 3907del | C | G | T | C |
| | q | C | T | G | A | 3907T | C | G | T | A |
| M-III-9 | Q | T | G | G | G | 3907del | C | G | T | C |
| | q | T | T | G | A | 3907T | C | G | T | A |
| L-II-16 | q | T | T | G | A | 3907T | C | G | T | A |
| | q | T | T | G | A | 3907T | C | G | C | A |
| M-I-1 | q | T | T | G | A | 3907T | C | G | T | A |
| | q | T | G | G | G | 3907T | C | G | C | A |
| N-II-1 | q | C | G | A | G | 3907T | T | A | T | C |
| | q | C | G | A | G | 3907T | T | A | T | C |
| N-II-4 | q | C | G | A | G | 3907T | T | A | T | C |
| | q | T | T | G | A | 3907T | C | G | T | A |

Since the bulls represented in this panel were born between 1952 and 1996 genetic trend has resulted in a significant increase in breeding value and trends in QTL allele frequency in time. To eliminate the possibility for bias due to time trends, an equation that is an estimate of one half of the Mendelian sampling of parental gametes was used in the analysis (M=PTA_{bull}−( 1/PTAₛᵢᵣₑ+½ PTA_{dam})). Consequently, M represented one-half of the deviation of the mean value of the two gametes inherited from the parents from the average of all possible parental gametes. The variance of the Mendelian sampling term will be larger in families that segregated for a major gene than those that were not segregating and the term was independent of the rate of genetic trend in a population. The M values were analyzed using ANOVA by contrasting animals that were heterozygous for the 3907T and 3907del alleles with animals that were homozygous for the 3907T allele (no animals were detected that were homozygous for the 3907del allele). The only SNP with a significant effect on any milk production trait was T3907del which influenced only PP (P=0.04).

To better estimate the frequency and effect of the T3907del SNP, 1,510 members of the super-family M (Table 1) except for families M-III-9 and M-III-12 were genotyped. Five families (M-II-1, M-III-10, M-IV-6, M-IV-8 and M-V-14) were also genotyped for the G3379T, G3490A and A3492G SNPs to construct haplotypes and test the effects of these polymorphisms. All of the sires (except M-III-12) of these families were homozygous for the 3907T allele at T3907del and the 3907del alleles present in their progeny were maternally inherited, allowing for the estimation of the effect of this SNP within the cow population. M values were analyzed using ANOVA as described above. Results and allele frequencies are shown in Table 5.

TABLE 5
Allele frequency and mean effect on PTA due to the four SNPs evaluated
within the OPN gene. P-values are presented under the estimated effects.
Haplotypes are in the order G3379T-G3490A-A3492G-T3907del.

| SNP | Class | N | MY | FY | FP | PY | PP |
|---|---|---|---|---|---|---|---|
| G3379T | GG | 147 | −46.5 | −2.91 | −0.0043 | −2.35 | −0.0043 |
| | GT | 493 | −44.6 | −2.56 | −0.0033 | −1.63 | −0.0015 |
| | TT | 362 | −86.1 | −1.85 | 0.0057 | −1.92 | 0.0025 |
| | f(G) = 0.39 | | 0.408 | 0.780 | 0.140 | 0.835 | 0.053 |
| G3490A | AA | 42 | −34.15 | 2.31 | 0.0157 | 1.87 | 0.0118 |
| | AG | 310 | −82.26 | −2.52 | 0.0024 | −1.91 | 0.0020 |
| | GG | 650 | −50.91 | −2.58 | −0.0024 | −2.05 | −0.0024 |
| | f(A) = 0.20 | | 0.583 | 0.225 | 0.208 | 0.165 | 0.005 |
| A3492G | AA | 361 | −84.13 | −1.80 | 0.0056 | −1.89 | 0.0024 |
| | AG | 496 | −43.77 | −2.53 | −0.0033 | −1.56 | −0.0013 |
| | GG | 145 | −54.81 | −3.13 | −0.0040 | −2.69 | −0.0047 |
| | f(G) = 0.39 | | 0.454 | 0.718 | 0.156 | 0.652 | 0.052 |
| T3907del | 3907del/3907T | 105 | 45.29 | −5.93 | −0.0318 | −3.70 | −0.0221 |
| | 3907T/3907T | 896 | −72.93 | −1.95 | 0.0036 | −1.64 | 0.0021 |
| | f(3907del) = 0.05 | | 0.014 | 0.031 | 1.36^{E}−06 | 0.124 | 6.62E−14 |
| Haplotype | GAG3907T | 180 | −110.85 | −1.92 | 0.0097 | −2.05 | 0.0047 |
| | GGG3907T | 144 | −26.72 | −2.77 | −0.0066 | −1.95 | −0.0046 |
| | GGG3907del | 83 | −7.60 | −7.34 | −0.0294 | −4.77 | −0.0202 |
| | TGA3907T | 584 | −60.65 | −1.72 | 0.0024 | −1.38 | 0.0017 |
| | | | 0.272 | 0.062 | 2.27E−04 | 0.172 | 3.27E−09 |

The OPN 3907del allele produced a 118.22 lb. increase in MY (P=0.014), 3.98 lb. decrease in FY (NS), 2.06 lb. decrease in PY (NS), 0.0354% decrease in FP (P=1.36E-6) and a 0.0242% decrease in PP (P=6.62E-14).

The G3490A SNP was significant for PP (P=0.005). This SNP can be excluded as being the causal QTN because segregating sires L-I-1, L-II-14 and M-II-9 were all homozygous for this SNP, and the association appeared to be due to linkage disequilibrium since the 3907del allele at T3907del occurred only in the haplotypes that harbor the G allele at G3490A (Table 5). Of the 45 evaluated sire families, 13 sires were heterozygous for G3490A. Of these 13 sires, 7 showed no evidence for segregation for any QTL in the vicinity of OPN, one was significant for a QTL centromeric of OPN (M-IV-8), two (N-II-6 and N-III-3) were significant for QTL near 67 cM, and three (L-II-15, L-II-17 and L-II-4) showed evidence of segregation for two QTL in the region (Table 3).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,916,779
U.S. Pat. No. 4,683,195
U.S. Pat. No. 4,683,202
U.S. Pat. No. 4,800,159
U.S. Pat. No. 4,883,750
U.S. Pat. No. 4,946,773
U.S. Pat. No. 5,279,721
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Patent Appl. 20040076977
Ashwell and Van Tassell, J. Dairy Sci., 82 (Suppl. 1):54, 1999.
Blott et al., Genetics, 163:253-266, 2003.
Churchill and Doerge, Genetics, 138:963-971, 1994.
D'Cruz et al., Molec. Endocrinology, 16:2034-2051, 2002.
Denhardt and Guo, FASEB, 7:1475-1482, 1993.
Drenkard et al., Plant Physiology, 124:1483-1492, 2000.
European Appln. 320 308
Freyer et al., J. Anim. Breed. Genet., 119:69-82, 2002.
Freyer et al., J. Dairy Sci., 86 (3):999-1008, 2003.
Frohman, In: PCR Protocols: A Guide To Methods And Applications, Academic Press, New York, 1990.
GB Appln. 2 202 328
Green et al., In: Documentation for CRI-MAP, version 2.4. Washington University School of Medicine, St. Louis, Mo., 1990.
Grisart et al., Genome Res., 12:222-231, 2002.
Grisart et al., Proc. Natl. Acad. Sci. USA, 101 (8):2398-2403, 2004.
Grisart et al., Proc. Natl. Acad. Sci. USA, 101 (8):2398-2403, 2004.
Heath, Am. J. Hum. Genet., 61:748-760, 1997.
Ihara, et al., Genome Res., 14:1987-1998.
Hillel et al., Genetics, 124 (3):783-789., 1990.
Kwoh et al., Proc. Natl. Acad. Sci. USA, 86:1173, 1989.
Ohara et al., Proc. Natl. Acad. Sci. USA, 86:5673-5677, 1989.
Olsen et al., J. Dairy Sci., 87:690-698, 2004.
PCT Appln. PCT/US87/00880
PCT Appln. PCT/US89/01025
PCT Appln. WO 88/10315
PCT Appln. WO 89/06700
PCT Appln. WO 90/07641
Ron et al., Genetics, 159:727-735, 2001.
Sambrook et al., In: Molecular cloning, Cold Spring Harbor Laboratory Press, Cold Spring Harbor, N.Y., 2001.
Schnabel et al., Cytogenetics Genome Res., 102:59-64, 2003.
Seaton et al., Bioinformatics, 18:339-340, 2002.
Spelman, et al., J. Dairy Sci., 85 (12):3514-3517, 2002.
Thallman et al., J. Anim. Sci., 79:26-33, 2001a.
Thallman et al., J. Anim. Sci., 79:34-44, 2001b.
Visscher et al., Genetics, 143:1013-1020, 1996.
Walker et al., Nucleic Acids Res., 20 (7):1691-1696, 1992.

What is claimed is:

1. A probe or primer comprising at least 15 contiguous nucleic acids of:
(a) the nucleic acid sequence of SEQ ID NO: 1 or a complement thereof; or (b) the nucleic acid sequence of SEQ ID NO: 1 further comprising at least one polymorphism at a nucleic acid base position selected from the group consisting of T1406C, G3379T, G3490A, A3492G, T3907del, C5075T, G5896A, T10043C, and A11740C, or a complement thereof.

2. An isolated nucleic acid molecule comprising the nucleic acid sequence of SEQ ID NO: 1 or the nucleic acid sequence of SEQ ID NO: 1 further comprising at least one polymorphism selected from the group consisting of T1406C, G3379T, G3490A, A3492G, T3907del, C5075T, G5896A, T10043C, and A11740C.

3. The nucleic acid molecule of claim 2, wherein the polymorphism is at position 3907 in SEQ ID NO: 1.

4. A method of determining the genetic predisposition of a bovine animal for altered milk production traits comprising genotyping the bovine to determine the genotype for OPN.

5. The method of claim 4, wherein genotyping is carried out by assaying of genetic material from the bovine.

6. The method of claim 4, wherein genotyping is carried out by PCR™.

7. The method of claim 4, wherein genotyping is carried out by nucleic acid hybridization.

8. The method of claim 4, wherein genotyping is carried out by determining the genotype of one or both of the parents of the bovine for OPN.

9. The method of claim 5, wherein the genetic material is from a gamete.

10. The method of claim 5, wherein the genetic material is genomic DNA.

11. The method of claim 2, comprising genotyping the bovine to determine the presence of at least one polymorphism in OPN selected from the group consisting of T1406C, G3379T, G3490A, A3492G, T3907del, C5075T, G5896A, T10043C, and A11740C.

12. The method of claim 10, wherein the polymorphism is T3907del.

13. The method of claim 4, wherein the altered milk production traits are an increase in milk yield, decrease in protein percentage, or decrease in fat percentage.

14. The method of claim 4, wherein the altered milk production traits are a decrease in milk yield, increase in milk protein percentage, or an increase in milk fat percentage.

15. A method of breeding dairy cattle having altered milk production traits, comprising the steps of:
(a) assaying at least one candidate head of dairy cattle to identify a first parent head of dairy cattle comprising a genetic polymorphism in OPN that confers altered milk production traits in female cattle comprising the polymorphism; and
(b) breeding the first parent head of dairy cattle with a second parent head of dairy cattle to obtain a progeny head of dairy cattle comprising the polymorphism.

16. The method of claim 15, wherein the altered milk production traits are an increase in milk yield, decrease in milk protein percentage, and decrease in milk fat percentage.

17. The method of claim 15, wherein the second parent head of dairy cattle comprises said genetic polymorphism.

18. The method of claim 15, further defined as comprising crossing said progeny head of dairy cattle with a third head of dairy cattle to produce a second generation progeny head of dairy cattle.

19. The method of claim 15, wherein said first parent head of dairy cattle is selected from a progeny head of dairy cattle resulting from a previous repetition of said step (a) and said step (b) and wherein said second parent head of dairy cattle is from a selected cattle breed into which one wishes to increase the occurrence of said polymorphism.

20. The method of claim 19, further defined as comprising repeating step (a) and step (b) from about 2 to about 10 times.

21. A method of breeding dairy cattle comprising:
(a) assaying a population of dairy cattle for the absence of a T3907del polymorphism in OPN in progeny of dairy cattle lacking the polymorphism;
(b) selecting members of the population lacking the T3907del polymorphism; and
(c) breeding the selected members of the population to produce progeny dairy cattle comprising the polymorphism.

22. A kit for detecting a polymorphism in the bovine OPN gene comprising first and second primers according to claim 1, the primers being complementary to nucleotide sequences of the OPN gene upstream and downstream, respectively, of a polymorphism in the bovine OPN gene which results in altered milk production traits.

23. The kit of claim 22, wherein the first primer comprises SEQ ID NO. 2.

24. The kit of claim 22, wherein the second primer comprises SEQ ID NO:3.
